(19) United States
(12) Patent Application Publication
(10) Pub. No.: US 2010/0016221 A1
(43) Pub. Date: Jan. 21, 2010

(54) METHOD OF DEGRADING PROTEIN BY CHAPERONE-MEDIATED AUTOPHAGY

(75) Inventors: Nobuyuki NUKINA, Wako-shi (JP); Peter BAUER, Wako-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi (JP)

(21) Appl. No.: 12/175,211

(22) Filed: Jul. 17, 2008

(51) Int. Cl. A61K 38/00; A61K 31/7088; C07K 14/00; C12N 15/11; C12N 15/00; A61P 43/00

(52) U.S. Cl. 514/12; 530/324; 536/23.5; 435/320.1; 514/44 R

(57) ABSTRACT

The present invention provides a method of degrading a target protein in a subject comprising administrating to the subject an effective amount of any of: (a) a peptide comprising an HSC70-binding moiety and a target protein-binding moiety; and/or (b) a polynucleotide encoding the peptide of (a).

The present invention further provides an isolated peptide comprising an HSC70-binding moiety and a target protein-binding moiety, an isolated polynucleotide encoding said peptide, and an expression vector comprising said polynucleotide.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 4,590 bytes ASCII (Text) file named “SequenceListing-703299.txt,” created Jul. 17, 2008.

### TECHNICAL FIELD OF THE INVENTION

The present invention relates to a novel method of degrading a target protein in a subject, such as an abnormal protein related to conformational diseases like Huntington's disease. The present invention also relates to an isolated peptide that can be used in the above method, a polynucleotide encoding said peptide, and an expression vector comprising said polynucleotide.

### BACKGROUND OF THE INVENTION

Huntington's disease (HD) is an autosomal-dominant neurodegenerative disorder caused by a CAG repeat expansion coding for polyglutamine (polyQ) in the N-terminal region of the huntington protein (htt) (see, H. Y. Zoghbi, H. T. Orr, Annu Rev Neurosci 23, 217 (2000)). A prominent feature of this disorder is progressive neurodegeneration, with the intranuclear and cytoplasmic accumulation of aggregated polyQ protein inside neurons (see, M. DiFiglia et al., Science 277, 1990 (1997), S. W. Davies et al., Cell 90, 537(1997)). Expanded polyQ forms a β-sheet structure, which causes formation of fibrillar and non-fibrillar aggregates (see, M. F. Perutz et al., Proc Natl Acad Sci USA 99, 5596 (2002), M. Tanaka et al., J Biol Chem 278, 34717 (2003)) and mediates aberrant interactions with transcription factors such as CBP, SP1, TAFII130 or NF—Y, disrupting the regulation of transcription (see, A. W. Dunah et al., Science 296, 2238 (2002), F. C. Nucifora, Jr. et al., Science 291, 2423 (2001), T. Yamanaka et al., EMBO J 27, 827 (2008)). While the pathological significance of the expanded polyglutamine has been clearly established, treatments that can actually prevent physical and mental decline associated with HD have not yet been developed, although several substances such as Congo red and trehalose have been reported to inhibit oligomerization or to stabilize the expanded polyQ proteins (see, ex., I. Sanchez et al., Nature 421, 373 (2003), M. Tanaka et al., Nat Med 10, 148 (2004)).

The therapeutic potential of down-regulating abnormal gene expression has been demonstrated in a tetracycline-regulated mouse model of HD (see, A. Yamamoto et al., Cell 101, 57 (2000)). Nuclear inclusions and behavioral abnormalities appeared with induction of the mutant htt expression. When expression in symptomatic mice was blocked, the inclusions disappeared and the behavioral phenotype was ameliorated, suggesting that therapeutic approaches aimed either at inhibition of mutant htt expression or its degradation might be effective. This is why many experimental treatments for polyQ diseases aim to decrease intracellular levels of the mutant protein without affecting the levels of the normal protein, which can be achieved either by decreased production or increased degradation of the mutant protein. Several techniques aiming to block htt expression have been explored, including small interfering RNAs (siRNAs) (see, ex., Y. L. Wang et al., Neurosci Res 53, 241 (2005)). Although human mutant gene expression could be specifically knocked down experimentally by siRNA against human htt without affecting endogenous normal mouse htt expression, specific inhibition of mutant gene expression might not be feasible due to ablation of the normal gene in humans. Enhancing the degradation of mutant protein is another therapeutic approach. Mutant htt is a substrate of proteasome, but the presence of expanded polyQ causes inhibition of the ubiquitin-proteasome system (UPS), which results in further accumulation of mutant htt (see, N. R. Jana et al., Hum Mol Genet 10, 1049 (2001), N. F. Bence et al., Science 292, 1552 (2001)). Autophagy is activated during UPS dysfunction perhaps in order to compensate for the reduced proteasome function, clearing up proteins not degraded by the UPS (see, U. B. Pandey et al., Nature 447, 859 (2007)). Attempts to increase the autophagic clearance of mutant htt resulted in reduced htt toxicity (see, S. Sarkar et al., Nat Chem Biol 3, 331 (2007)), however, the hyperactivation of macroautophagy may be deleterious to the cell. Thus, there is a demand for another method of efficiently reducing aggregated polyQ protein in a cell, wherein the method relies on a different mechanism from those mentioned above, and may potentially lead to the development of treatment/prophylaxis of related diseases.

### SUMMARY OF THE INVENTION

The object of the present invention, therefore, is to provide a novel method of degrading a target protein in a subject, especially abnormal protein associated with diseases. To achieve the object, the present inventors have exploited the ability of chaperone-mediated autophagy (CMA) to selectively degrade specific substrates. Specific chaperones such as the heat-shock cognate protein of 70 kDa (Hsc70), bind to target proteins containing a specific sequence and channel them to the surface of the lysosome where they bind to lysosome associated membrane protein 2a (Lamp2a). The target protein is then transported across the lysosomal membrane and degraded by vacuolar proteases (A. M. Cuervo, J. F. Dice, Science 273, 501 (1996), F. A. Agarraberes, F. Dice, J Cell Sci 114, 2491(2001)). It has been reported that α-synuclein is degraded by CMA and that the signal sequence for this degradation is the HSC-binding motif VKKDQ (related to the consensus sequence KFERQ) (A. M. Cuervo et al., Science 305, 1292 (2004)). The present inventors introduced a molecule containing a combination of HSC70-binding motifs and a glutamine-binding peptide to a HD model mouse, and found that it resulted in a significant decrease of polyQ aggregation, ameliorated symptoms and prolonged the lifespan of the model mouse. This suggests that the technique may be applicable in reducing the level of abnormal protein by CMA in various diseases, and the utilization of CMA can have a promising potential in the treatment/prophylaxis of such diseases.

The present inventors have conducted further investigations based on the above findings, and completed the present invention.

Accordingly, the present invention provides:

[1] A method of degrading a target protein in a subject comprising administrating to the subject an effective amount of any of the following (a) and (b):

(a) a peptide comprising an HSC70-binding moiety and a target protein-binding moiety;

(b) a polynucleotide encoding the peptide of (a);

[2] The method according to above [1], wherein the HSC70-binding moiety comprises at least one motif capable of binding with HSC70;

[3] The method according to above [2], wherein the motif(s) is the amino acid sequence of SEQ ID NO:1 and/or the amino acid sequence of SEQ ID NO:2;

[4] The method according to above [1], wherein the target protein is an abnormal protein;

[5] The method according to above [4], wherein the abnormal protein is involved in a conformational disease;

[6] The method according to above [5], wherein the conformational disease is a polyglutamine disease;

[7] The method according to above 6, wherein the polyglutamine disease is Huntington's disease;

[8] The method according to above [1], wherein the target protein-binding moiety comprises a Polyglutamine-binding peptide 1 (QBP1);

[9] The method according to above [1], wherein the subject is a mammal;

[10] An isolated peptide comprising an HSC70-binding moiety and a target protein-binding moiety, wherein the target protein is an abnormal protein;

[11] An isolated polynucleotide encoding the peptide of above [10];

[12] The polynucleotide according to above [11], wherein the abnormal protein is involved in a conformational disease;

[13] The polynucleotide according to above [12], wherein the conformational disease is a polyglutamine disease;

[14] The polynucleotide according to above [13], wherein the polyglutamine disease is Huntington's disease;

[15] The polynucleotide according to above [11], wherein the target protein-binding moiety comprises a Polyglutamine-binding peptide 1 (QBP1);

[16] An expression vector comprising the polynucleotide of above [11] operably linked to a promoter;

[17] The vector according to above [16], wherein the vector is a viral vector.

The method of the present invention enables the selective reduction of target protein level in a subject by enhancing the degradation of said protein, which shows advantageous effect over prior art such as in the amelioration of Huntington's disease in a model mouse.

These or other characteristics and the advantages of the present invention will be apparent from the detailed description of the invention below.

### BRIEF DESCRIPTION OF THE DRAWINGS

FIG. 1 shows RHQ inhibits polyQ aggregation more efficiently than RQ. (A) Constructs (R, mRFP; RS, mRFP-scrambled(QBP1)₂; RHS, mRFP-HSC70bm-scrambled(QBP1)₂; RQ, mRFP-(QBP1)₂; RHQ, mRFP-HSC70bm-(QBP1)₂). (B) Confocal images of the 150Q Neuro2a cells transfected with tested constructs. Molecules containing (QBP1)₂ co-localized with the polyQ inclusions (bar, 10 μm). (C) The expression of RQ and RHQ in 150Q Neuro2a cells markedly decreased the polyQ aggregation (gel top). (D) Quantification revealed 38% and 56.5% decreases of inclusion formation in 150Q Neuro2a cells by RQ and RHQ, respectively, after 24 hours of differentiation and induction. (E) In 150Qnls Neuro2a cells, decrease of the polyQ inclusion formation by 21.5% (RQ) and 48.5% (RHQ) was observed after 48 hours of differentiation and induction. Values in (D) and (E) represent mean±SEM from four independent experiments (*P<0.05, **P<0.005). The quantifications were performed by ArrayScan.

FIG. 2 shows (R)HQ induces the degradation of expanded polyQ through CMA. (A) Western blot analysis of the chase experiment in 60Q Neuro2a cells. (B) RHQ enhanced the 60Q degradation during the chase period. Values represent relative mean±SD from three independent experiments (*P<0.05). Value of R represents the control condition (R=1). (C, D) Tested constructs without mRFP (schemes of S, HS, Q, HQ are in FIG. 6A) were co-transfected with (C) tNhtt-16Q-EGFP (bar, 4 μm) and (D) tNhtt-150Q-EGFP construct to Neuro2a cells (bar, 8 μm). (E) Dot blot analysis of the purified lysosomes from Neuro2a cells co-transfected with tNhtt-60Q and tested constructs. (PNS, post-nuclear supernatant). (F) Quantification of htt translocation into lysosomes. Levels of htt in PNS were normalized to β-tubulin and in lysosomes to cathepsin D (CathD). (G) Compound images generated by ArrayScan illustrating inclusion formation in 150Q Neuro2a cells transfected with tested constructs and shRNA for HSC70 and/or Lamp2a. (blue, Hoechst 33258; green, polyQ-EGFP; red, tested molecule). (H) RNA interference of HSC70, Lamp2a or both alleviated the inhibitory effect of RHQ on polyQ inclusion formation. Values in (F) and (H) represent mean±SEM from three independent experiments (*P<0.05, ***P<0.001).

FIG. 3 shows the effect of the rAAV-HQ in R6/2 mouse brains. (A) FTA analysis of polyQ aggregation in nine R6/2 mouse brains injected with three different combinations of rAAV. (B) Quantification of the SDS-insoluble material detected by FTA. (C) Western blot analysis of htt aggregation (gel top) and higher molecular polyQ complexes by AGERA method. (D) Quantification of the gel top-detected aggregation. (E) Confocal images of infected striatal neurons from three mice infected with different rAAV combinations (bar, 5 μm). (F) Brain sections stained with anti-RFP, EM48 and anti-ubiquitin antibodies (magnification, 40×). (G) Count of ubiquitin-positive inclusions in the transduced areas. Bars in (B), (D) and (G) represent the relative mean values±SEM from three brains injected with different rAAV in each striatum. R in R/Q and R/HQ, and Q in Q/HQ brains represent the control value of 1. (*P<0.05, **P<0.005, ***P<0.001).

FIG. 4 shows the effect of rAAV-HQ on R6/2 mice phenotype. (A) Body weight changes in mice with bilateral injection at 6-14 weeks of age (n=11 for all three groups until 12 weeks; at 14 weeks, n=7 for R/R, n=10 for Q/Q, n=11 for HQ/HQ). (B) Clasping scores. (C) Rotarod performance; n=11 for all three groups (*P<0.05). (D) Survival curve. Median survival (days): R/R-108; Q/Q-123; HQ/HQ-140. Mean survival (days): R/R-105.5±3.9; Q/Q-122.3±4.8; HQ/HQ-136.2±4.4. Log-Rank test, P<0.0001; Wilcoxon test, P<0.0001.

FIG. 5 shows presence of HSC70bm within the polyQ protein decreases its aggregation. (A) Scheme of tNhtt-60Q-Venus with inserted HSC70bm sequences. (B) PC12 cell transfected with two different polyQ constructs. The HSC70bm caused decreased inclusion formation of the polyQ protein and diffuse Venus fluorescence in many cells (bar, 20 μm). (C) Quantification of the PC12 cells with inclusions 24 hours after transfection. The cells were grown under normal (ser+) or serum withdrawal (ser−) conditions. (D) Macroautophagy was blocked by 10 mM 3MA, main lysosomal proteases cathepsin D and E were inhibited by 10 μM pepstatin A, and cathepsin A was inhibited by 10 μM leupeptin. Values in (C) and (D) represent mean±SEM of four independent experiments. (*P<0.05, **P<0.005). The quantifications were performed by ArrayScan.

FIG. 6 shows the effect of the linker molecule on polyQ aggregation and cytotoxicity in inducible 150Q and 150Q-nls Neuro2a cells. (A) Schemes and abbreviations of used constructs. (B) Inclusion formation in 150Q Neuro2a cells. (C) Inclusion formation in 150Q-nls Neuro2a cells. (D) Cell death in 150Q Neuro2a after 60 hours of induction. (E) Cell death in 150Q-nls Neuro2a cells after 48 hours of induction. Values in (B), (C), (D) and (E) represent mean±SEM of four independent experiments. (*P<0.05, **P<0.005, ***P<0.001). The quantifications were performed by ArrayScan.

FIG. 7 shows HQ enhanced the degradation of polyQ protein in 150Q and tNhtt-62Q kikGR Neuro2a cells. (A) Western blot analysis of the chase experiment in 150Q Neuro2a cells after 24 hours. (B) Chase experiment using the tNhtt-62Q-kikGR Neuro2a cell line. The photo-cleaved form of 62Q-kikGR was chased for 12 hours. Upper panel shows the full length 62Q-kikGR after short exposure. Longer exposure was needed for visualization of the cleaved form. (C) Quantification of the 62Q kikGR clearance by optical density (OD) of the anti-HA bands normalized to P-tubulin. Bars represent the relative mean values±SEM from three independent experiments. RQ values represent the control conditions (RQ=1). (*P<0.05, **P<0.005).

FIG. 8 shows RT-PCR analysis of the HSC70 and Lamp2a silencing in Neuro2a cells by shRNA. For further experiments, shRNAs used in underlined lanes were used.

FIG. 9 shows activation of CMA by 300 nM geldanamycin enhanced the effect of RHQ on intracellular levels of tNhtt-60Q-EGFP. (A) Western blot analysis. (B) Quantitative analysis of the polyQ levels in left panel of (A). (C) Quantitative analysis of the polyQ levels in right panel of (A). Values in (B) and (C) represent mean OD of anti-GFP normalized by P-tubulin levels±SEM from three independent experiments. (*P<0.05, ***P<0.001).

FIG. 10 shows distribution of rAAV-HQ in the R6/2 mouse brain 4 weeks after the intrastriatal injection, direct fluorescence in fresh frozen saggital section. Especially neostriatum and medial globus pallidus appear to be transducted in this mouse. Transduction was also observed in the part of cerebral cortex and globus pallidus (STR, striatum; CTX, cerebral cortex; LV, lateral ventricle) (bar, 600 μm).

FIG. 11 shows injection of rAAV to the HD190Q-EGFP mouse brain. (A) Example of the distribution of rAAV-HQ 10 weeks after the intrastriatal injection, direct fluorescence in fresh frozen coronal section. The virus transduction was distributed throughout the whole striatum. Magnification, 4×. (B) Effect of rAAV-R, -Q and -HQ on polyQ aggregation in the transduced areas of the striata. Magnification, 8×. (C) Inclusions count in the transduced area of the paired striata. Bars represent the relative mean values±SEM from three brains injected with different rAAV. R in R/Q and R/HQ, and Q in Q/HQ brains represent the control value of 1.

### DETAILED DESCRIPTION OF THE INVENTION

The present invention provides a novel peptide that induces protein degradation through chaperone-mediated autophagy, and a polynucleotide encoding the peptide, wherein the peptide comprises (a-i) an HSC70-binding moiety and (a-ii) a target protein-binding moiety.

(a-i) An “HSC70-binding moiety” means a peptide moiety which comprises at least one HSC70-binding motif that is recognized by and binds to HSC70. The HSC70-binding motif includes KFERQ-related motifs which are known to serve as signals for targeting proteins for lysosomal proteolysis via CMA. Examples of KFERQ-related motifs include, but are not limited to, peptides having amino acid sequences shown by SEQ ID NOs:1 (KFERQ), 2 (VKKDQ), 3 (EFLKQ), 4 (QKVFD), 5 (QELRR), and 6 (QEFIK), preferably SEQ ID NOs:1 and 2. HSC70-binding motifs can also have an amino acid sequence in which 1, 2, or 3 amino acids are substituted by or added to the amino acid sequences of SEQ ID NOs:1-6, as long as the motifs retain the HSC70-binding ability. HSC70-binding moiety may comprise one or more (eg., 1 to 5, preferably 1 to 3, more preferably 2) HSC70-binding motifs, wherein the plurality of motifs may consist of the same motif, or consist of a combination of different motifs. In a preferable embodiment, the HSC70-binding moiety comprises the amino acid sequence of SEQ ID NO:1 and/or the amino acid sequence of SEQ ID NO:2.

HSC70-binding ability can be assayed by general methods known in the art such as the two-hybrid method, co-immunoprecipitation method, protein chip analysis (ex. SPR chips), phage-display method, and Far-Western blot analysis.

(a-ii) A “target protein-binding moiety” means a peptide moiety which enables the peptide to recognize and selectively bind to a target protein. The target protein is preferably an abnormal protein involved in a disease, more preferably involved in a conformational disease.

“Conformational diseases” means a group of disorders sharing a striking similarity in molecular mechanisms, in which various diseases categorized as conformational diseases, each arise from an aberrant conformational transition in an underlying protein, which lead to protein aggregation, and as a result cause tissue deposition (see, Carrell et al., 1997). Examples of conformational diseases include cystic fibrosis, polyglutamine diseases, prion-related diseases, Alzheimer's disease, antitrypsin deficiency, and systemic amyloidosis. In the present invention, the conformational disease is preferably a polyglutamine disease, which are inherited brain disorders caused by an expanded CAG (amino acid sequence encoding glutamine) repeat in the patient's disease gene, leading to the toxic accumulation of mutant expanded polyQ protein in neurons. Examples of polyglutamine diseases include Huntington's disease, Kennedy's disease, dentatorubropallidoluysian atrophy, Machado-Joseph disease and spinocerebellar ataxias.

When the target protein of the present invention is involved in a polyglutamine disease, the target protein-binding moiety of the peptide of the present invention preferably comprises a polyglutamine binding moiety. The polyglutamine binding moiety comprises at least one amino acid sequence capable of binding with polyglutamine. A preferable example of such amino acid sequence is Polyglutamine-binding peptide 1 (QBP1) shown by the amino acid sequence of SEQ ID NO:7 (SNWKWWPGIFD). QBP1 is a tryptophan-rich peptide identified from a combinatorial peptide library which binds preferentially to expanded polyglutamine, and inhibits its aggregation in an in vitro assay (Y. Nagai et al., J Biol Chem 275, 10437(2000)). Other examples of polyglutamine-binding sequences may include polyglutamine-binding domains of known polyglutamine-binding proteins such as PQBP-1 to -4, and the like.

Polyglutamine binding moieties can also comprise an amino acid sequence in which 1, 2, 3, 4 or 5 amino acids are deleted from, substituted by or added to the amino acid sequences of SEQ ID NO:7, as long as the sequence retains the polyglutamine-binding ability. The polyglutamine binding ability can be assayed according to the above-mentioned methods used for assaying the HSC70-binding ability.

Polyglutamine binding moiety may comprise one or more polyglutamine-binding sequences. When the moiety comprises two or more polyglutamine-binding sequences, these sequences may be the same or different.

The peptide of the present invention can be obtained by a method known per se. For example, the peptide can be obtained by (i) culturing a host cell transformed with a polynucleotide that encodes the peptide (see below) and recovering the peptide from the culture broth, (ii) biochemical synthesis using a cell-free protein synthesis system such as rabbit reticulocyte lysate system, wheat germ lysate system, or E. coli lysate system, or (iii) chemical synthesis such as solid-phase synthesis. Alternatively, when the target protein-binding moiety is, for example, a naturally occurring protein capable of binding with the target protein, the peptide of the present invention may also be obtained by isolating the protein from a cell or tissue producing the same using known protein separation techniques, and coupling it to an HSC70-binding motif using any cross-linking reagent such as those used when crosslinking a hapten to a carrier protein, and the like. Examples of cross-linking reagents include diazonium compounds such as bisdiazobenzidine, dialdehyde compounds such as glutaraldehyde, dimaleimide compounds, and the like. The peptide thus produced can be separated and purified by methods such as chromatography (ex. reversed-phase chromatography, ion exchange chromatography, or affinity chromatography), salt or solvent precipitation, dialysis, ultrafiltration, gel filtration, SDS-PAGE, electrofocusing, or combinations thereof.

The polynucleotide provided by the present invention is not limited as long as it encodes the peptide of the present invention described above, preferably a polynucleotide comprising a nucleotide sequence encoding the amino acid sequence of QBP1 (SEQ ID NO:7) as a target protein-binding moiety and the amino acid sequences of tandemly arranged two HSC70-binding motifs (i.e., SEQ ID NOs: 1 and 2) as an HSC70-binding moiety. More preferably, the polynucleotide of the present invention comprises the nucleotide sequence shown by SEQ ID NO:8 (ATGGCCCGAGTTAAGAAGGATCAAGCTGAGCCGCTGCACCGAAAGTTCGAACGTCAACCGC CCGGGTCGAACTGGAAGTGGTGGCCAGGTATCTTCGACTCGAACTGGAAGTGGTGGCCAGGT ATCTTCGAC), as well as a polynucleotide that comprises a nucleotide sequence hybridizing to the nucleotide sequence shown by SEQ ID NO:8 under stringent conditions and encoding a peptide capable of binding with both HSC70 and a target protein.

As examples of the polynucleotide capable of hybridizing to the nucleotide sequence shown by SEQ ID NO:8 under stringent conditions, DNA that comprises a nucleotide sequence showing a homology of about 70% or more, preferably about 80% or more, more preferably about 90% or more, particularly preferably about 95% or more, and most preferably about 97% or more, to the nucleotide sequence shown by SEQ ID NO:8, can be used.

Hybridization can be conducted according to a method known per se or a method based thereon, for example, a method described in Molecular Cloning, 2nd edition (J. Sambrook et al., Cold Spring Harbor Lab. Press, 1989) and the like. When a commercially available library is used, hybridization can be conducted according to the method described in the instruction manual attached thereto. Hybridization can preferably be conducted under highly stringent conditions.

High-stringent conditions refer to, for example, conditions involving a sodium concentration of about 19 to 40 mM, preferably about 19 to 20 mM, and a temperature of about 50 to 70° C., preferably about 60 to 65° C. In particular, a case wherein the sodium concentration is about 19 mM and the temperature is about 65° C. is preferred.

The polynucleotide of the present invention can be obtained by synthesizing its full length nucleotide sequence by methods known per se, such as by using a commercially available DNA/RNA synthesizer (Applied Biosystems, Beckman, etc.), or isolating both or either of a polynucleotide encoding the HSC70 binding moiety and a polynucleotide encoding the target protein-binding moiety from any cell or tissue expressing the same, and ligating both polynucleotide using known recombinant DNA techniques.

The present invention also provides an expression vector in which the above-described polynucleotide operably linked to a promoter has been inserted thereinto. By “operably linked to a promoter”, is meant that the polynucleotide is linked to the promoter so that the promoter allows the polynucleotide to be transcribed.

The backbone of the expression vector of the present invention include viral vectors and plasmid vectors, preferably viral vectors, but is without limitation as long as the polypeptide of the present invention is expressed in a given host. Examples of viral vectors include adenoviral, retroviral, lentiviral, adeno-associated viral, herpes viral, vaccinia viral, pox viral, polioviral, Sindbis viral, and Sendai viral vectors, which are all preferable vectors for administration to mammals.

The promoter may be any promoter that can function in a given cell into which the polynucleotide of the present invention is to be introduced, and include viral promoters such as SRα promoter, SV40 early promoter, CMV immediate early promoter, RSV promoter, and MoMuLV promoter, as well as mammalian constitutive promoters such as β-actin promoter, PGK promoter, and transferrin promoter.

The expression vector of the present invention may further comprise elements such as sites for initiation or termination of transcription, ribosome binding site in the transcription region necessary for translation, posttranscriptional regulatory elements such as WPRE, polyadenylation sequences, replication origin, and selectable marker genes such as drug-resistant genes.

As mentioned above, the present invention provides a method for degrading a target protein, which comprises administrating to a subject an effective amount of the above-described peptide or the polynucleotide.

The subject of the present invention can be any organism, as long as it requires the reduction of a target protein level within its body, and is preferably an animal, more preferably a mammal (for example, human, chimpanzee, mouse, rat, rabbit, sheep, pig, cow, horse, cat, dog, and the like), even more preferably a human, chimpanzee, dog, mouse, or rat, and most preferably a human.

The peptide or the polynucleotide (desirably inserted into an appropriate expression vector) can be mixed with a pharmacologically acceptable carrier required to yield a pharmaceutical composition, and then administered to the subject.

As examples of the pharmacologically acceptable carrier, various organic or inorganic carrier substances conventionally used as pharmaceutical preparation materials can be mentioned, and these are formulated as excipients, lubricants, binders and disintegrants, in solid preparations; as solvents, solubilizing agents, suspending agents, isotonizing agents, buffering agents and soothing agents, in liquid preparations, and the like. Also, as necessary, pharmaceutical preparation additives such as antiseptics, antioxidants, coloring agents, sweeteners and the like can be used.

As examples of suitable excipients, lactose, saccharose, D-mannitol, D-sorbitol, starch, gelatinized starch, dextrin, crystalline cellulose, low substituted hydroxypropyl cellulose, sodium carboxymethyl cellulose, gum arabic, pullulan, light silicic anhydride, synthetic aluminum silicate, magnesium metasilicate aluminate and the like can be mentioned.

As examples of suitable lubricants, magnesium stearate, calcium stearate, talc, colloidal silica and the like can be mentioned.

As examples of suitable binders, gelatinized starch, sucrose, gelatin, gum arabic, methyl cellulose, carboxymethyl cellulose, sodium carboxymethyl cellulose, crystalline cellulose, saccharose, D-mannitol, trehalose, dextrin, pullulan, hydroxypropyl cellulose, hydroxypropylmethyl cellulose, polyvinyl pyrrolidone and the like can be mentioned.

As examples of suitable disintegrants, lactose, saccharose, starch, carboxymethyl cellulose, calcium carboxymethyl cellulose, sodium crosscarmellose, sodium carboxymethyl starch, light silicic anhydride, low substituted hydroxypropyl cellulose and the like can be mentioned.

As examples of suitable solvents, water for injection, physiological saline, Ringer's solutions, alcohols, propylene glycol, polyethylene glycol, sesame oil, corn oil, olive oil, cottonseed oil and the like can be mentioned.

As examples of suitable solubilizing agents, polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like can be mentioned.

As examples of suitable suspending agents, surfactants such as stearyl triethanolamine, sodium lauryl sulfate, lauryl aminopropionic acid, lecithin, benzalkonium chloride, benzethonium chloride and glyceryl monostearate; hydrophilic polymers such as polyvinyl alcohol, polyvinyl pyrrolidone, sodium carboxymethyl cellulose, methyl cellulose, hydroxymethyl cellulose, hydroxyethyl cellulose and hydroxypropyl cellulose; polysorbates, polyoxyethylene hardened castor oil and the like can be mentioned.

As examples of suitable isotonizing agents, sodium chloride, glycerin, D-mannitol, D-sorbitol, glucose and the like can be mentioned.

As examples of suitable buffers, buffer solutions of a phosphate, an acetate, a carbonate, a citrate and the like, and the like can be mentioned.

As examples of suitable soothing agents, benzyl alcohol and the like can be mentioned.

As examples of suitable antiseptics, paraoxybenzoates, chlorobutanol, benzyl alcohol, phenethyl alcohol, dehydroacetic acid, sorbic acid and the like can be mentioned.

As examples of suitable antioxidants, sulfides, ascorbates and the like can be mentioned.

As examples of suitable coloring agents, water-soluble food tar colors (e.g., food colors such as Food Red Nos. 2 and 3, Food Yellow Nos. 4 and 5, and Food Blue Nos. 1 and 2), water-insoluble lake pigments (e.g., aluminum salts of the aforementioned water-soluble food tar colors and the like), natural pigments (e.g., β-carotene, chlorophyll, red iron oxide and the like) and the like can be mentioned.

As examples of suitable sweeteners, sodium saccharide, dipotassium glycyrrhizinate, aspartame, stevia and the like can be mentioned.

As examples of dosage forms of the aforementioned pharmaceutical composition, oral formulations such as tablets, capsules (including soft capsules and microcapsules), granules, powders, syrups, emulsions and suspensions; non-oral formulations such as injections (e.g., subcutaneous injections, intravenous injections, intramuscular injections, intraperitoneal injections and the like), external formulations (e.g., nasal preparations, transdermal preparations, ointments and the like), suppositories (e.g., rectal suppositories, vaginal suppositories and the like), pellets, drops, sustained-release preparations (e.g., sustained-release microcapsules and the like) and the like can be mentioned; these can be safely administered orally or non-orally.

The pharmaceutical composition can be produced by a method conventionally used in the field of pharmaceutical preparation making, for example, a method described in the Japanese Pharmacopoeia and the like. A specific method of producing a preparation is hereinafter described in detail. The content of the peptide or polynucleotide in the pharmaceutical composition varies depending on the dosage form, the dose of the compound and the like; and is, for example, from about 0.1 to 100% by weight.

For example, an oral formulation is produced by adding to an active ingredient an excipient (e.g., lactose, saccharose, starch, D-mannitol and the like), a disintegrant (e.g., calcium carboxymethyl cellulose and the like), a binder (e.g., gelatinized starch, gum arabic, carboxymethyl cellulose, hydroxypropyl cellulose, polyvinyl pyrrolidone and the like), a lubricant (e.g., talc, magnesium stearate, polyethylene glycol 6000 and the like) and the like, compression-molding the resultant mixture, and subsequently, as required, coating the resulting material with a coating base by a method known per se for the purpose of taste masking, enteric solubility or sustained release.

As examples of the coating base, a sugar-coating base, a water-soluble film coating base, an enteric film coating base, a sustained-release film coating base and the like can be mentioned.

As the sugar-coating base, saccharose is used, which may be used in combination with one species or two or more species selected from among talc, precipitated calcium carbonate, gelatin, gum arabic, pullulan, carnauba wax and the like.

As examples of the water-soluble film coating base, cellulose polymers such as hydroxypropyl cellulose, hydroxypropylmethyl cellulose, hydroxyethyl cellulose and methylhydroxyethyl cellulose; synthetic polymers such as polyvinylacetal diethylanimoacetate, aminoalkylmethacrylate copolymer E [Eudragit-E (trade name), Rohm Pharma Corp.] and polyvinyl pyrrolidone; polysaccharides such as pullulan; and the like can be mentioned.

As examples of the enteric film coating base, cellulose polymers such as hydroxypropylmethyl cellulose phthalate, hydroxypropylmethyl cellulose acetate succinate, carboxymethylethyl cellulose, and cellulose acetate phthalate; acrylic polymers such as Methacrylic Acid Copolymer L [Eudragit-L (trade name), Rohm Pharma Corp.], Methacrylic Acid Copolymer LD [Eudragit-L-30D55 (trade name), Rohm Pharma Corp.], and Methacrylic Acid Copolymer S [Eudragit-S (trade name), Rohm Pharma Corp.]; natural substances such as shellac, and the like can be mentioned.

As examples of the sustained-release film coating base, cellulose polymers such as ethyl cellulose; acrylic polymers such as aminoalkyl methacrylate copolymer RS [Eudragit-RS (trade name), Rohm Pharma Corp.], and an ethyl acrylate-methylmethacrylate copolymer suspension [Eudragit-NE (trade name), Rohm Pharma Corp.]; and the like can be mentioned.

The above-mentioned coating bases may also be used in a mixture of two or more kinds thereof in a suitable ratio. Also, during coating, a shading agent, for example, titanium oxide or iron sesquioxide, may be used.

An injection is produced by dissolving, suspending or emulsifying an active ingredient in an aqueous solvent (e.g., distilled water, physiological saline, Ringer's solution and the like), an oily solvent (e.g., vegetable oils such as olive oil, sesame oil, cottonseed oil and corn oil, propylene glycol, and the like), or the like, along with a dispersing agent (e.g., polysorbate 80, polyoxyethylene hydrogenated castor oil 60, polyethylene glycol, carboxymethyl cellulose, sodium alginate and the like), a preservative (e.g., methylparaben, propylparaben, benzyl alcohol, chlorobutanol, phenol and the like), an isotonizing agent (e.g., sodium chloride, glycerin, D-mannitol, D-sorbitol, glucose and the like), and the like. At this time, if desired, additives such as a solubilizing agent (e.g., sodium salicylate, sodium acetate and the like), a stabilizer (e.g., human serum albumin and the like), a soothing agent (e.g., benzyl alcohol and the like) and the like may also be used. An injection solution is normally packed in an appropriate ampoule.

The dosage of the peptide or the polynucleotide varies depending on the subject of administration, route of administration, the severity of the disease, and the like; in an adult human patient infected with Huntington's disease (body weight 60 kg), for example, the dosage is about 0.001 mg to 5 g, preferably about 0.1 to 500 mg, more preferably about 1.0 to 20 mg, per day, based on the active peptide administrated to the subject.

The present invention is hereinafter described in more detail by means of the following examples, which, however, are not to be construed as limiting the present invention.

### Experimental Procedures

### (1) Mice.

Two HD mouse models were used in the experiment. Heterozygous htt exon 1 transgenic female mice of the R6/2 strain (see, S. W. Davies et al., supra) (145 CAG repeats; Jackson code, B6CBATgN (HD exon 1) 62) were originally obtained from Jackson Laboratory (Bar Harbor, Me.). The HD 190Q-EGFP transgenic female mice harbor mutant truncated N-terminal htt containing 190 CAG repeats fused with EGFP in its genome. This animal shows progressive motor abnormality, and neuropathology such as formation of inclusions in brain, and shorter lifespan (S. Kotliarova et al., J. Neurochem. 93, 3 (2005)). All the experiments with mice were approved by the Animal Experiment Committee of the RIKEN Brain Science Institute.

### (2) Materials.

The autophagy inhibitor 3-methyladenine (3MA) was purchased from Sigma. The cathepsin D and E inhibitor pepstatin A and cathepsin L inhibitor leupeptin were from Nacalai tesque and geldanamycin was from Wako Chemicals. Hoechst 33258 and Lysotracker-rhodamine were obtained from Molecular Probes. Mouse monoclonal antibody specific for N-terminal of htt (EM48) and rat monoclonal anti-β-tubulin antibodies were from Chemicon. Anti-GFP and anti-RFP antibodies were from MBL. Anti-ubiquitin was purchased from Dako, anti-cathepsin D and anti-HA were from Santa Cruz Biotechnology.

### (3) Plasmids.

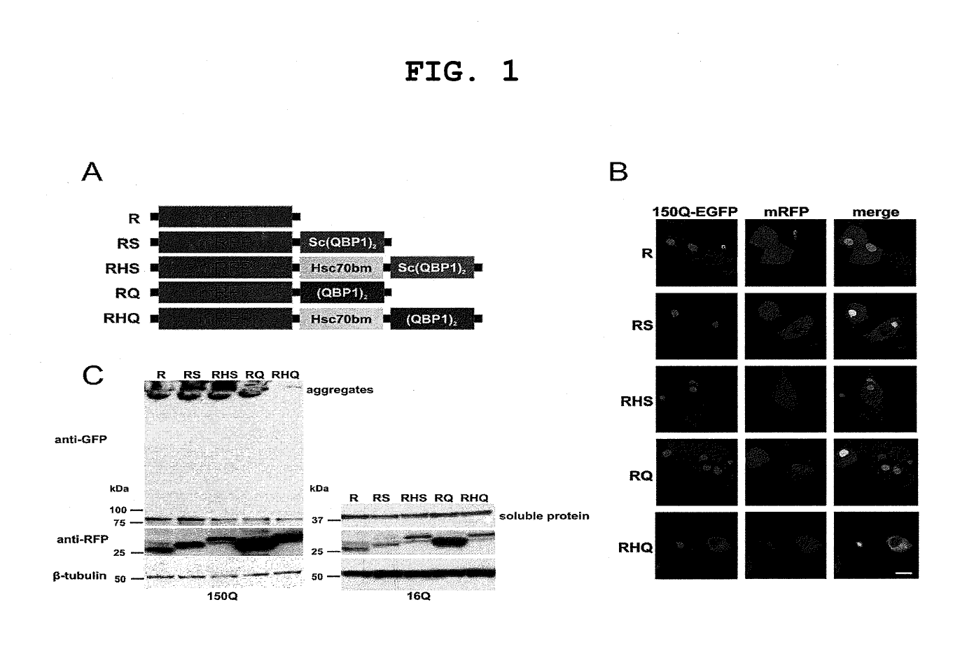

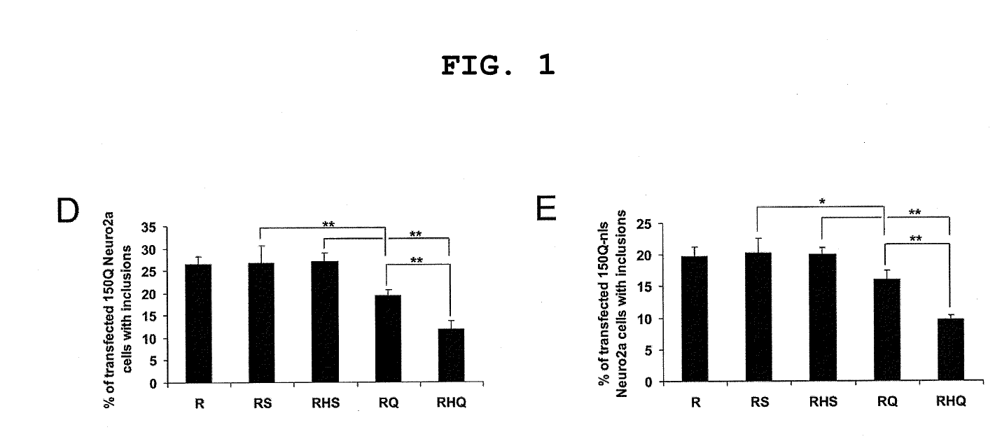

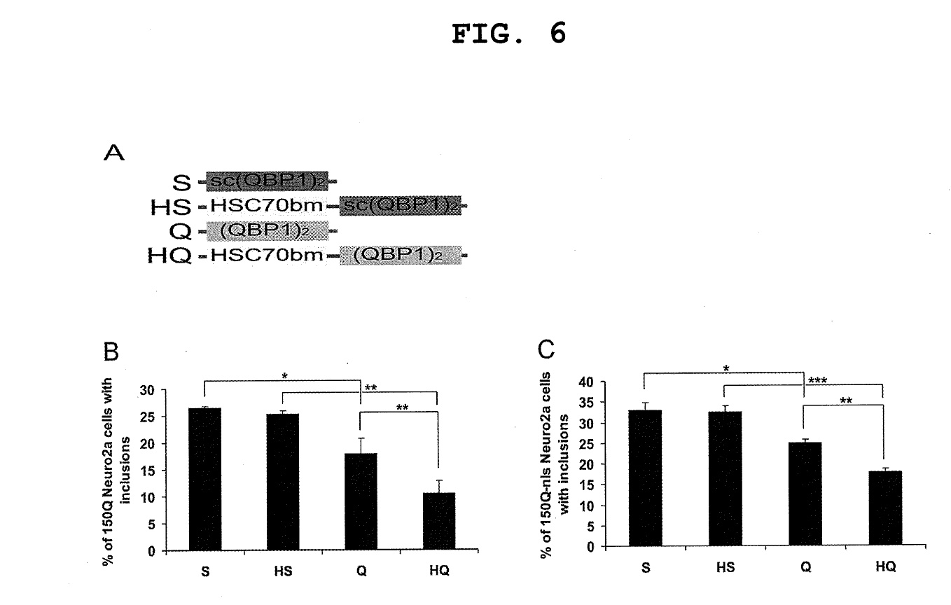

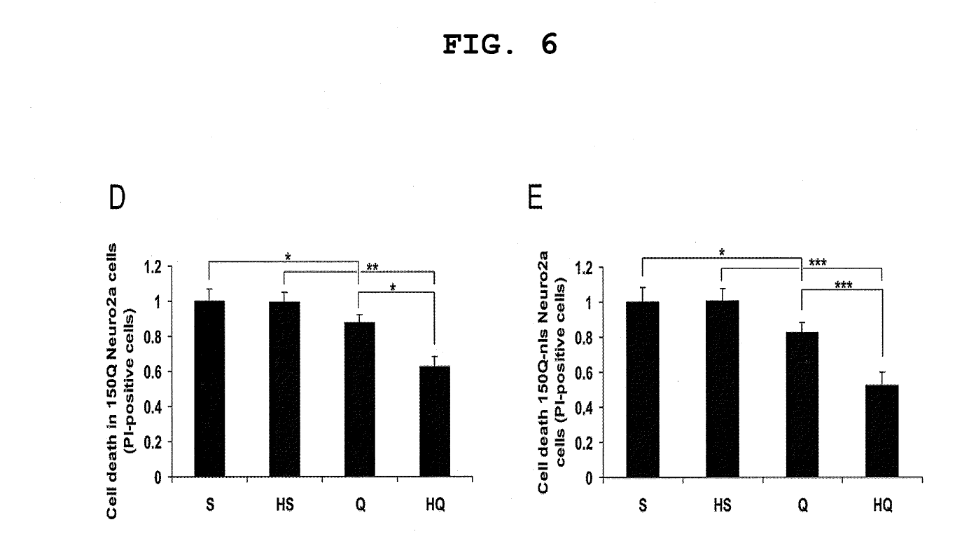

The construction of the N-terminal fragment of human huntington exon 1 (tNhtt) encoding 60 CAG repeats was previously described (G. H. Wang et al., Neuroreport 10, 12(1999)). This fragment was fused to the N-terminal of a variant of yellow fluorescent protein (Venus) (T. Nagai et al., Nat. Biotechnol. 20, 1 (2002)) and inserted into the pcDNA3.1 vector (60Q). Two Hsc70 binding motifs (HSC70bm) (SEQ ID NOs:9 and 10: in bold) were inserted between the Cfr13I and BamHI cutting sites of 60Q: 5′-GTTAAGAAGGATCAA-GCTGGAGCCGCTGCACCG-AAGTTCGAACGTCAA-3′ (SEQ ID NO:11). The tNhtt-16Q-EGFP and tNhtt-150Q-EGFP plasmids for transient transfection were prepared by the introduction of the coding sequence into pcDNA3.1 vector (Invitrogen). Oligonucleotides encoding the active and scrambled (QBP1)₂ were synthesized (Operon), annealed, fused to C-terminus of monomeric red fluorescent protein (mRFP) and inserted into the pcDNA3.1 vector. HSC70bm were amplified with primers containing SalI in the forward and XmaI cutting site in the reverse primer from the 60QHsc construct. The PCR product was introduced between mRFP and scrambled or active (QBP1)₂. To produce molecules without mRFP tag, all constructs were amplified using primers containing BamHI in the forward and XbaI in the reverse primer, respectively, and introduced to pcDNA3.1 vector. The schemes and abbreviations of the constructs are listed in FIGS. 1A and 6A. The amino acid sequences of the expressed molecules are displayed in Table 1 (without mRFP tag).

TABLE 1

Names, abbreviations and amino-acid sequences
of the tested molecules. The HSC70bm are in
bold and QBP1 underlined.

| | | | |
|---|---|---|---|
| SEQ ID NO:12 | Scrambled | S | MWGWPNDFDWKGWFSPWKISWIN |
| | (QBP1)₂ | | |
| SEQ ID NO:13 | (QBP1)₂ | Q | MSNWKWWPGIFDSNWKWWPGIFD |
| SEQ ID NO:14 | HSC70bm- | HS | MARVKKDQAEPLHRKFERQPPGWG |
| | scrambled | | WPNDFDWKGWFSPWKISWIN |
| | (QBP1)₂ | | |
| SEQ ID NO:15 | HSC70bm- | HQ | MARVKKDQAEPLHRKFERQPPGSN |
| | (QBP1)₂ | | WKWWPGIFDSNWKWWPGIFD |

Monomeric KikGR (mKikGR) was kindly gifted from Dr. Miyawaki, RIKEN BSI Japan. KikGR is cleaved and photo-converted by irradiation of ˜350-420nm (H Tsutsui et al., supra). It contains additional eleven mutations in the KikGR tetramer (BAD95669), such as A17S, G32R, I37T, N39T, C116T, V126T, N161E, Q167E, H219Y, L222T and P223Y. The pFRT-KikGR was constructed by insertion of mKikGR fragment PCR-applied using a specific primer set, 5′-CCGAATTCATGCTAGCACCATGGATCCTAGTGTGATTACATCAGAAATG-3′ (SEQ ID NO:16) as forward and 5′-TTTTAGATCTTATCCGGACTTGGCTTCAAATTCATACTTGGCGCC-3′ (SEQ ID NO:17) as reverse primer, into NheI and BamHI sites of pcDNA5/FRT/TR vector (Invitrogen). The pFRT-tNhtt-62Q-KikGR was then generated by inserting the htt exon 1 with 62Q (HD62) (H. Doi et al., FEBS Lett. 571, 3-1 (2004)) into EcoRI and BamHI sites of pFRT-mKikGR vector. pFRT-HD62-HA-KikGR was finally constructed by the introduction of HA epitope into the BamHI site using following double-stranded oligo DNA, GATCTATACCCATACGATGTTCCAGATTACGCG (SEQ ID NO:18) and GATCCGCGTAATCTGGAACATCGTATGGGTATA (SEQ ID NO:19). All constructs were verified by sequencing.

### (4) Cell Culture, Transient Transfection and Treatments.

Mouse neuroblastoma (Neuro2a) cells were maintained in Dulbecco's modified Eagle's medium (Sigma) supplemented with 10% heat-inactivated fetal bovine serum (Sigma), 100 U/ml penicillin and 100 μg/ml streptomycin (Invitrogen) at 37° C. in an atmosphere containing 5% CO₂ and 95% air. Establishment of stable Neuro2a cell lines with the ecdysone-inducible mammalian expression system (Invitrogen), that express tNhtt-EGFP-16Q (16Q Neuro2a cells), tNhtt-EGFP-150Q (150Q Neuro2a cells) and tNhtt-EGFP-150Q-nls (150Q-nls Neuro2a cells) has been described earlier (T. Nagai et al., supra, H. Doi et al., supra, E. A. Zemskov et al., J Neurochem. 87, 2 (2003)). Neuro2a cells were differentiated with 5mM dbcAMP (N⁶, 2′-O-dibutyryladenosine-3′,5′-cyclic monophosphate sodium salt) (Nacalai Tesque) and induced to express tNhtt-polyQ with 2 μM ponasterone A (ponA; Invitrogen) for indicated times. Neuro2a/FRT/TR cell line was generated as described in the protocol for Flp-in/T-Rex system (Invitrogen). Neuro2a tNhtt-62Q-kikGR cell line was generated by transfecting Neuro2a/FRT/TR cells with the pFRT-HD62-KikGR and pOG44 (Invitrogen) constructs using Lipofectamine 2000 reagent and selected with 200 μg/ml hygromycin. The expression of the pFRT-tNhtt-62Q-KikGR was induced by 18 doxycycline treatment of the cells. For the photo-induced kikGR cleavage, cells were exposed to ˜400 nm wavelength for 5 minutes. Rat pheochromocytoma (PC12) cells were grown in the same conditions as Neuro2a cells, except for the serum composition of 5% of fetal bovine and 10% of horse serum (Sigma). All transient transfections were performed with Lipofectamine 2000 (Invitrogen) according to the manufacturer's instruction.

### (5) Cell Death Assay.

For quantification of cell death, 5 μg/ml each of Hoechst 33342 and propidium iodide (PI) were added to differentiated and induced Neuro2a cells transiently transfected with either of the tested constructs (FIG. 6A). After 10 minutes at 37° C., the number of cells with PI uptake over the total number of cells was calculated by ArrayScan.

### (6) Isolation of Intact Lysosomes.

We used a modification of the previously described method for the purification of lysosomes from CHO cells (E. A. Madden, B. Storrie, Anal. Biochem. 163, 2 (1987), E. A. Madden, J. B. Wirt, B. Storrie, Arch. Biochem. Biophys. 257, 1 (1987)). Neuro2a cells grown in 10 cm dishes were co-transfected with tNhtt-60Q and tested constructs, and after 16 hrs of incubation, cells were washed with ice cold PBS and homogenized by ten strokes. The homogenate was centrifuged at 1300 g for 5 min. The supernatant was decanted and placed on ice. The nuclear fraction was resuspended in 0.25 M sucrose and centrifuged at 1300 g for 5 min. This step was repeated 3 times and the supernatants from each wash were pooled to give a total post-nuclear supernatant (PNS). All the solutions used for the preparation of the discontinuous gradient were in 0.25 M sucrose. Gradients were prepared in cellulose nitrate tubes for Beckman SW40 rotor. Two ml of 35% Histodenz (Sigma) was overlaid with 2 ml of 17% Histodenz followed by 5 ml of 6% Percoll (Sigma). The tubes were then filled with 4.8 ml of PNS. Centrifugations were performed at 50,500 g for 15 min in Beckman Optima TLX ultracentrifuge. After the first centrifugation, 1.125 ml of the material from the 6% Percoll/17% Histodenz interface was mixed with 0.875 ml of 80% Histodenz and placed in the bottom of a new SW40 tube and overlaid by 2 ml of 17% Histodenz followed by 2 ml of 5% Histodenz. The tubes were then gently filled with 0.25 M sucrose and centrifuged again. The 5%/17% Histodenz interface was highly enriched in lysosomes.

### (7) RNA Interference.

Each sense and anti-sense template short hairpin (sh) RNA for Lamp2a and HSC70 was purchased from Operon, annealed and ligated into pSilencer1.0 vector with U6 promotor according to the manufacturer's instructions (Ambion). The target sequences were as follows:

(SEQ ID NO:20)

| | |
|---|---|
| Lamp2a, | 5′-AACCATTGCAGTACCTGACAA-3′; |

(SEQ ID NO:21)

| | |
|---|---|
| HSC70, | 5′-AACTGGAGAAAGTCTGCAACC-3′. |

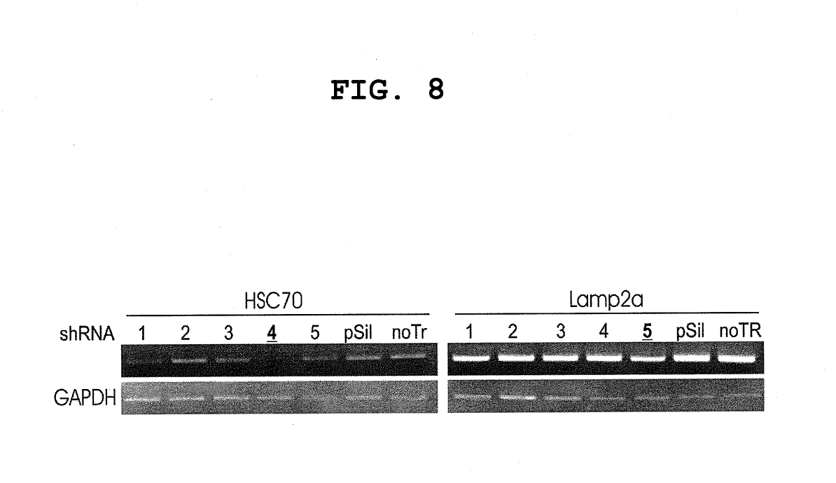

The plasmids containing shRNA were sequence-verified. Plasmids were transfected into Neuro2a cells using Lipofectamine 2000. After 2 days of silencing, cells were differentiated and induced. The inventors performed RT-PCR to verify the knockdown efficiency (FIG. 8).

### (8) ArrayScan Quantification.

For the quantification of the inclusions, cells were grown in 24-well plates for indicated periods, fixed in 4% paraformaldehyde, washed and incubated with Hoechst 33258 at 1/1000 dilution in PBS. Cells were analyzed by ArrayScan® V^{TI} High Content Screening (HSC) Reader (Cellomics) using Target Activation BioApplication (TABA). TABA analyzes images acquired by a HSC Reader and provides measurements of the intracellular fluorescence intensity and localization on a cell-by-cell basis.

In each well, at least 10000 cells were counted and quantified for the presence of the inclusions. Nuclei stained with Hoechst 33285 provided the autofocus target and their count gave the exact number of the quantified cells. The screening itself consisted of two scans using Hoechst, FITC (for GFP) and TRITC (for RFP) fluorescence. Firstly, number of inclusions in transfected cells was calculated when fluorescent spots at size of at least 5 pixels (magnification 20× for cytoplasmic and 40× for nuclear aggregates) with average GFP intensity more than 1500 in the RFP background were counted. Secondly, nuclei were defined as the objects of interest and the cells with average intensity more than 50 within 3 pixels from the nucleus were selected for the analysis. The percentage of the cells with aggregates was then calculated. When the constructs without mRFP were used for transfection, the procedure was same, except the RFP intensity was not measured. Scanning was performed with triplicate or quadruplicate in each experimental condition. Data was generated from the quantification of more than 250,000 cells in each experimental set-up.

### (9) Chase Experiments.

To determine whether tNhtt-polyQ degrades faster in the presence of RHQ, chase experiments were performed. Neuro2a were first induced to express tNhtt-polyQ for 20 hrs in case of 60Q and 12 hrs in case of 150Q cells and then transfected with the tested constructs. Four hours later, ponaA was removed; cells were washed with PBS and incubated in the medium containing dbcAMP (for differentiation) for 24 hrs. Cells were lysed and the levels of tNhtt-polyQ were analyzed by Western blotting.

Neuro2a tNhtt-62Q-kikGR cells were induced for 24 hours, and then the 62Q-kikGR was cleaved by 5 minutes irradiation of the cells. Chase phase lasted for 12 hours before the cells were collected and analyzed.

(10) Construction and Stereotaxic Injection of rAAV-R, -Q and -HQ.

The viral expression constructs rAAVl/2-CAG-(R; Q; HQ)-WPRE were prepared by subcloning of mRFP (R), mRFP-(QBP1)₂ (Q) and mRFP-HSC70bm-(QBP1)₂ (HQ) into an adeno-associated (serotype-2) viral (rAVE™) cassette which is flanked by the AAV inverted terminal repeats (ITR). The viral cassette contained a hybrid CMV enhancer/chicken P-actin promoter (CAG), a woodchuck posttranscriptional regulatory element (WPRE),and a bovine growth hormone (BGH) polyadenylation sequence. Viral vectors were packaged and affinity purified (GeneDetect) for high expression in mouse brain tissue. The stereotaxic injections of rAAV into R6/2 mouse striata were performed at age of 4 weeks. The animals were first anesthetized by intraperitoneal injection of pentobarbital and placed in a stereotaxic apparatus. rAAV were injected into the right and left striatum through burr holes in the skull using a 5 μL Hamilton syringe mounted on the stereotaxic apparatus. Injections were placed 0.5 mm anterior to the bregma, 1.5 mm lateral to the sagittal suture and 2 mm below the skull surface. The rate of injection was 0.3 μl/min with total volume of 3 μl (equivalent to 3.6×10⁹ genomic particles). Mice were sacrificed at 8 weeks of age and the level of aggregation in the striatum was analyzed by Western blot, filter trap assay and immunohistochemistry. Another group of R6/2 mice was injected bilaterally with same rAAV for phenotype analysis. HD190Q-EGFP mice were injected at age of 6 weeks and the striata analyzed 10 weeks later.

### (11) Immunoblotting.

Cells were washed twice with ice-cold PBS, scraped, and resuspended in lysis buffer (0.5% Triton X-100 in PBS, 0.5 mM phenylmethylsulfonyl fluoride, Complete protease inhibitor mixture (Roche Applied Sciences)). After incubating on ice for 30 minutes lysates were briefly sonicated. Equal amounts of protein were boiled for 5 minutes in 2× SDS-sample buffer, separated by 5-12% gradient SDS-PAGE and electrophoretically transferred to a polyvinylidene difluoride (PVDF) membrane (Millipore). The membranes were blocked in 5% skim milk in 0.05% Tween 20/Tris-buffered saline (TBST) and incubated with primary antibody (dilutions in accordance to manufacturer's recommendations) overnight at 40° C. Then the membranes were washed three times in TBST and incubated for 1 hour with horseradish peroxidase-conjugated secondary antibody (dilution 1:5000). Immunoreactive proteins were detected with enhanced chemiluminescence reagents (Amersham Biosciences). Dot blots were prepared by blotting 10 μg of total proteins onto nitrocellulose membranes using a vacuum manifold and were processed as described for Western blots.

### (12) Filter Trap Assay (FTA).

FTA was performed using a Hybri-Dot manifold (BIORAD) and cellulose acetate membrane filter with pore size of 0.2 μM (Advantec). The cell lysates were prepared as for Western blotting. Same amount of protein from each experimental condition was diluted to 100 μl in PBS with 2% SDS and applied onto the membrane. Soluble proteins were removed by vacuum suction while the SDS-resistant aggregates stayed trapped. Wells were washed three times with 2% SDS/PBS and suction was maintained for 20 minutes to allow complete and tight trapping of SDS in soluble material. Membranes were subsequently blocked with 5% skim milk and immunoblot was performed.

### (13) Agarose Gel Electrophoresis for Resolving Aggregates (AGERA).

AGERA is a simple and sensitive biochemical detection method for quantitative and qualitative investigations of aggregate formation in HD models (A. Weiss et al., J. Neurochem. 104, 3 (2008)). Briefly, 1.5% agarose gels (BMBio) were prepared in buffer 375 mmol/L Tris-HCl₁, pH 8.8 buffer and brought to boiling in a microwave oven. After melting, SDS was added to a final concentration of 0.1%. Gels were poured on trays resulting in a gel thickness of 8 mm. Samples were diluted in 2× non-reducing Laemmli sample buffer (150 mmol/L Tris-HCl pH 6.8, 33% glycerol, 1.2% SDS and bromophenol blue) and incubated for 5 min at 95° C. After loading, gels were run in Laemmli running buffer (192 mmol/L glycine, 25 mmol/L Tris-base, 0.1% SDS). Semi-dry electroblotter Trans-blot SD Cell (Biorad) was used to blot the gels on PVDF membranes at 150 mA per 10 cm² for 1.5 hour in the transfer buffer containing 192 mmol/L glycine, 25 mmol/L Tris-base, 0.1% SDS, and 15% methanol. A 0.5 kg weight was centered on the electroblotter's top to guarantee constant and even contact between the gel and the electroblotter when blotting these gels. After transfer, starting with the blocking step, immunoblot membranes were processed as described in Western blot.

### (14) Histology.

Serial-cut 20-micrometer sections were used for immunohistochemistry after fixation in 4% paraformaldehyde. Sections were treated with anti-RFP and EM48 antibodies followed by AlexaFluor568-labelled anti-rabbit and AlexaFluor433-labeled anti-mouse secondary antibodies (Molecular Probes). For calorimetric detection, antibodies against RFP, ubiquitin and htt (EM48) were used followed by detection using ABC Elite kit (Vector Laboratories). For direct RFP and GFP fluorescence, frozen sections were used.

(15) The In Vivo Study of rAAV-HQ in Female R6/2 Mice.

To address the beneficial effect of (R)HQ in vivo, the inventors employed the R6/2 mouse model in which the progressive HD pathology is well characterized and has been extensively used for pre-clinical drug testing (M. F. Beal et al., supra).

Viral vectors encoding R, Q and HQ were injected to the left and right striatum at 4 weeks of age. Body weight was measured every second week starting from 4 to 14 weeks of age. The clasping and rotarod performance was tested at the time of body weight measure starting from 4 and 6 weeks, respectively. For the clasping score, mice were suspended by the tail for 30 seconds and the clasping phenotype was graded to a particular level according to the following scale: 0—no clasping; 1—clasping of the forelimbs only; 2—clasping of both fore and hind limbs once or twice; 3—clasping of both fore and hind limbs more than 3 times or more than 5 seconds.

Before each rotarod testing, mice were first trained on a rotating rod moving at 4 rpm for 5 min. The testing itself was performed on the rotating rod with linearly increasing speed from 4 rpm up to 45 rpm in 300 seconds. Mice of 6 weeks to 12 weeks of age were all subjected to rotarod test with the same moving speed. For the survival distribution, the number of days each mouse survived was recorded and the data collected (R/R, n=11; Q/Q, n=11; HQ/HQ, n=11) were subjected to Kaplan-Meier analysis followed by log-rank testing.

### (16) Statistical Analysis.

The inventors used unpaired student's t-test for comparison between two sample groups. One-way ANOVA Fisher's test followed by Tukey's HSD test was used for multiple comparisons with a 95% confidence level. These data were generated with XLSTAT software. For survival rate, the survival distribution curve was plotted with the Kaplan-Meier method followed by log-rank and Wilcoxon testing (JMP Statistical Discovery software, SAS Institute). The difference between comparisons was considered to be significant when P<0.05 for all the statistical analysis.

### EXAMPLE 1

Effect of HSC70bm and (QBP1)₂ on Aggregation of polyQ and polyQ-Related Cytotoxicity

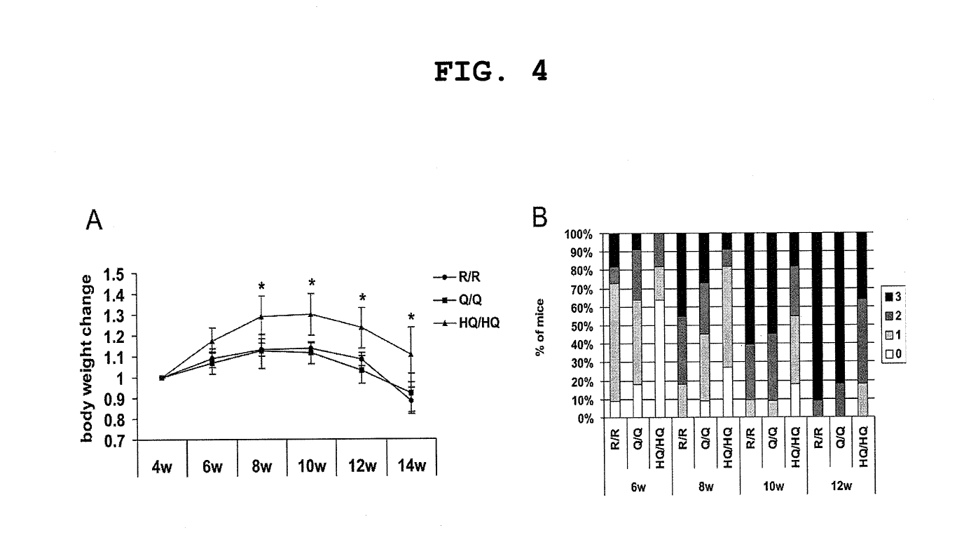

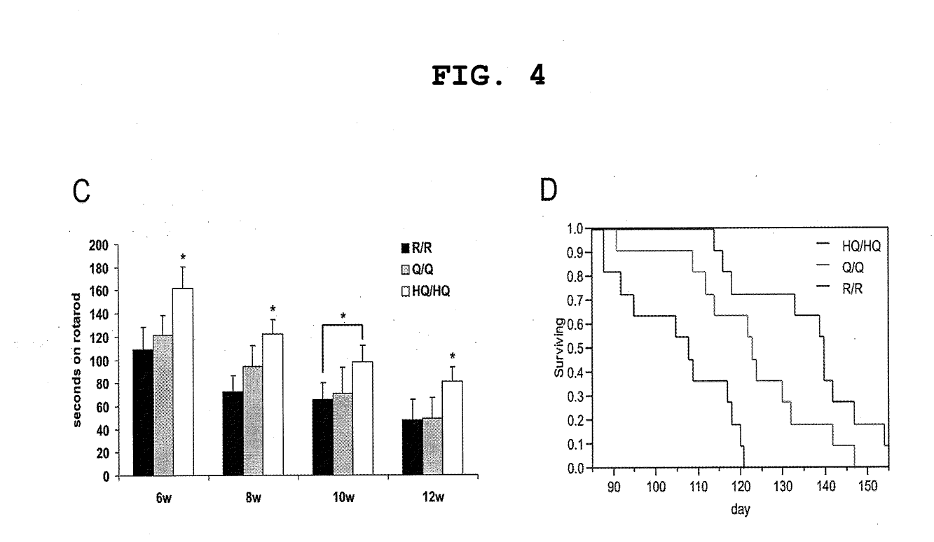

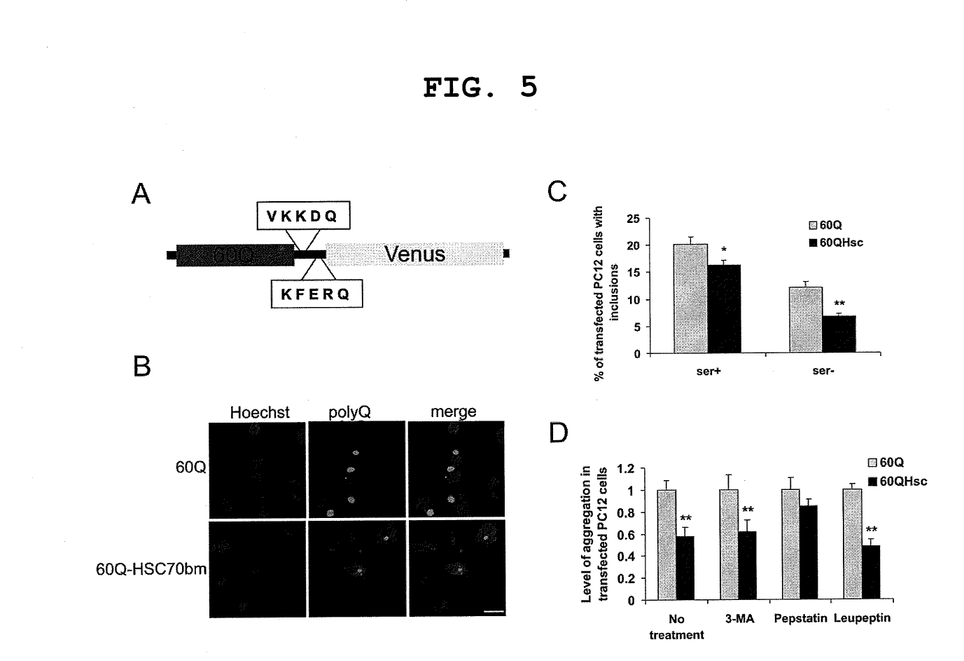

The present inventors introduced two HSC70 binding motifs (HSC70bm) into truncated N-terminal htt with 60Q-Venus (60QHsc) (FIG. 5A) and expressed 60Q and 60QHsc in PC12 cells (FIG. 5B). The constructs containing the HSC70bm generated fewer aggregates and this decrease was more pronounced when autophagy was activated by serum withdrawal (FIG. 5C). Blocking lysosomal proteases cathepsin D and E by pepstatin A alleviated the effect of HSC70bm while macroautophagy inhibition by 3-methyladenine (3MA) did not (FIG. 5D). Interestingly, the inhibition of cathepsin A that negatively regulates Lamp2a levels (see, A. M. Cuervo, et al., EMBO J 22, 47 (2003)), accentuated the difference in aggregation between 60Q with and without HSC70bm (FIG. 5D). These data suggested that the presence of HSC70bm enhanced the degradation of expanded polyQ protein by CMA. The present inventors therefore decided to investigate whether a protein or peptide linking polyQ and HSC70 would be able to induce the degradation of expanded polyQ through CMA. To test this, the present inventors designed a molecule containing two HSC70bm (H) and the duplicated sequence of the previously reported Polyglutamine Binding Peptide 1 (QBP1)₂ which was shown to bind specifically to the expanded but not normal polyQ and inhibit aggregation by preventing its oligomerization (Y. Nagai et al., Hum Mol Genet 12, 1253 (2003), Y. Nagai et al., J Biol Chem 275, 10437 (2000)). The present inventors explored the effect of this molecule on polyQ expression and whether it could degrade the polyQ protein, and the present inventors performed comparisons with the constructs listed in FIG. 1A (molecules conjugated with monomeric Red fluorescent protein; mRFP; R). When transfected to a stable Neuro2a cell line with inducible expression of tNhtt-150Q, the (QBP1)₂-containing molecules (RQ and RHQ; FIG. 1A) co-localized with the polyQ inclusions (FIG. 1B) and decreased the polyQ aggregation whereas RHQ had much stronger effect (FIG. 1C). The levels of tNhtt-16Q ininducible Neuro2a cells were not affected by RQ or RHQ transfection (FIG. 1C). Inclusions counting also revealed that RHQ had a stronger inhibitory effect on polyQ aggregation compared to RQ in 150Q (FIG. 1D), and 150Qnls Neuro2a cells (FIG. 1E). To investigate the effect of those molecules on polyQ-related cytotoxicity, the present inventors used the constructs without mRFP to enable the utilization of a fluorescent marker for cell toxicity, propidium iodide (PI) (FIG. 6A). The 150Q and 150Qnls Neuro2a cells were transfected and later induced and differentiated for indicated times for the cell death assay. The (QBP1)₂ (Q) and HSC70bm-(QBP1)₂ (HQ) constructs were able to decrease the aggregation and number of PI-positive cells compared to their control counterparts, scrambled(QBP1)₂ (S) and HSC70m-scrambled(QBP1)₂ (HS), respectively in both cell lines (FIG. 6, B-E). Specifically, 150Q Neuro2a cells, 4 hrs after transfection, were induced and differentiated for 24 hours, then fixed and analyzed for aggregation. Both Q and HQ were able to decrease the aggregation in 150Q cells compared to their control counterparts (S and HS), by 32.6% and 58.5%, respectively (FIG. 6B). In 150Q-nls Neuro2a cells 48 hours after induction, Q reduced the aggregation by 24.7% and HQ by 45.2% (FIG. 6C). Cell death assay in 150Q Neuro2a after 60 hours of induction showed that Q decreased the percentage of PI-positive cells by 12.3% and HQ by 37.4% (FIG. 6D). Furthermore, cell death assay in 150Q-nls Neuro2a cells after 48 hours of induction showed that Q decreased the percentage of PI-positive cells by 17.7% and HQ by 47.2% (FIG. 6E). As a result HQ decreased the 150Q and 150Qnls aggregation and cytotoxicity far more efficiently than did Q.

### EXAMPLE 2

Molecule Comprising HSC70bm and (QBP1)₂ (RHQ) Enhances polyQ Degradation

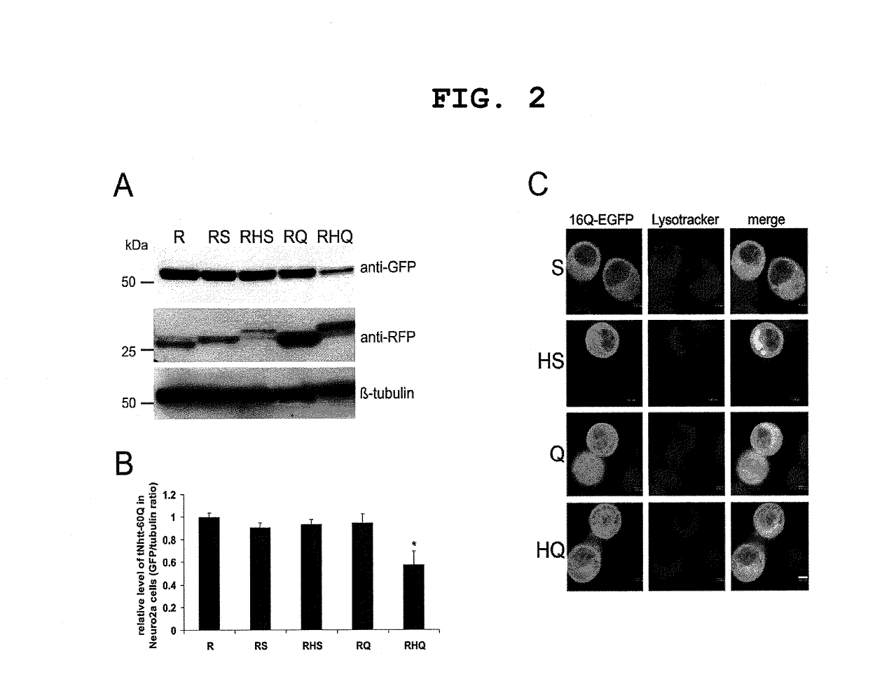

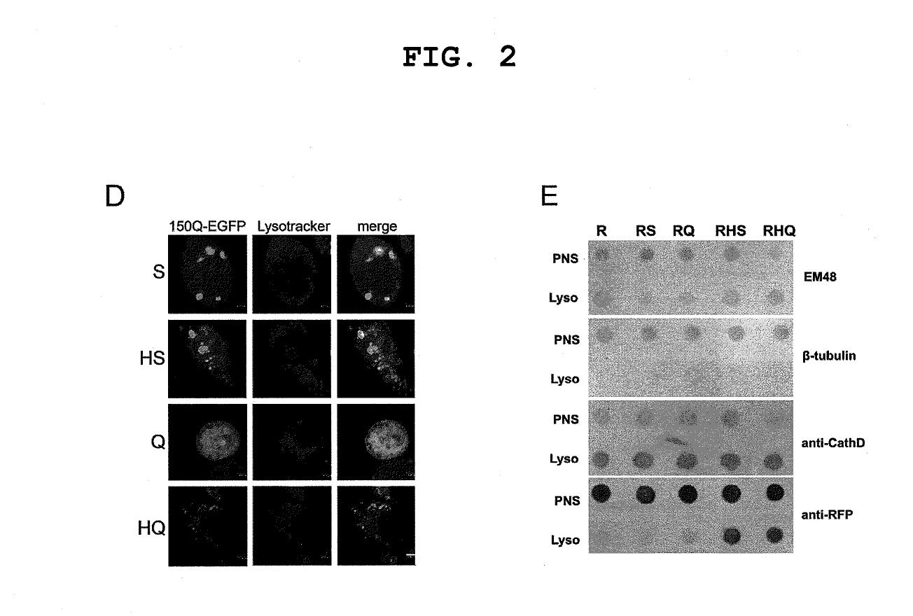

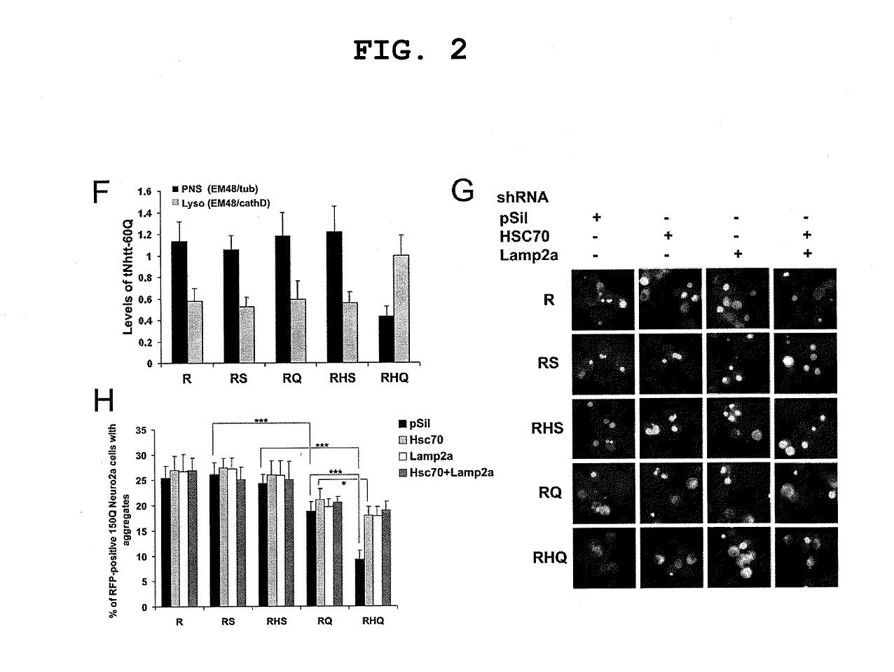

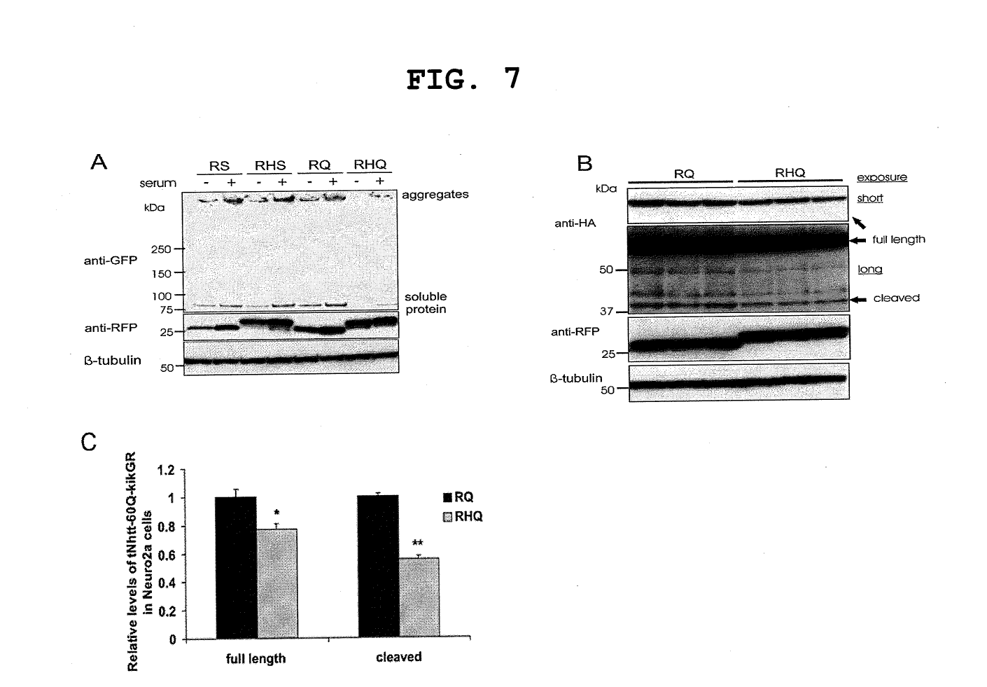

Next, the present inventors examined the mechanistic platform for the enhanced inhibition of the polyQ aggregation by RHQ. It has been shown that QBP1 inhibits toxic conformational transition of the expanded polyQ stretch, which is thought to be a trigger for polyQ protein oligomerization and aggregation (Y. Nagai et al., Hum Mo, Genet 12, 1253 (2003), Y. Nagai et al., Nat Struct Mol Biol 14, 332 (2007)). This probably leads to enhanced accessibility of the mutant protein to degradation systems. Since the addition of HSC70bm intensified the effect of (QBP1)₂ on polyQ aggregation and protein levels, the present inventors examined and compared the rate of degradation by RQ and RHQ. The present inventors performed a chase experiment using 60Q Neuro2a cells and found that RHQ enhanced the degradation of the soluble tNhtt-60Q protein by 43% as compared to R and by 37.4% as compared with RQ after 24 hours (FIGS. 2,A and B). An experiment using 150Q Neuro2a cells also showed enhanced degradation of the polyQ protein by RHQ (FIG. 7A). Autophagy activation by serum withdrawal caused more pronounced degradation with almost complete clearance of aggregated and soluble polyQ when co-expressed with RHQ (FIG. 7A). To confirm the enhanced degradation of the polyQ protein, the present inventors used the tNhtt-62Q-kikGR Neuro2a cell line system, where kikGR is cleaved after irradiation with wavelength ˜400 nm (H. Tsutsui, S. Karasawa, H. Shimizu, N. Nukina, A. Miyawaki, EMBO Rep 6, 233 (2005)). As the cleaved form of the protein is produced at a certain time point, its level is a good indicator of protein degradation. Twelve hours after irradiation, RHQ was able to decrease the levels of the cleaved form of tNhtt-62Q-kikGR protein by 45.6% while the full length (constitutively expressed) showed a 24% decrease as compared to RQ (FIGS. 7,B and C). These results clearly demonstrated the effectiveness of RHQ in the enhancement of tNhtt-polyQ degradation.

### EXAMPLE 3

RHQ Targets polyQ to Lysosomes

To investigate whether the (R)HQ targets the polyQ to lysosomes, the present inventors co-transfected HQ or other constructs with 16Q-EGFP or 150Q-EGFP to Neuro2a cells for 16 hours. In the cells expressing 16Q-EGFP, none of the co-transfected constructs had any effect on the subcellular distribution of the green fluorescence (FIG. 2C). On the other hand, when 150Q-EGFP was co-expressed with HQ, fluorescence intensity decreased, redistributed and partly co-localized with the lysosomal marker Lysotracker-rhodamine (FIG. 2D). To confirm this observation, the present inventors co-transfected the tested constructs with tNhtt-60Q to Neuro2a cells, 16 hours later purified intact lysosomes and analyzed them for the presence of the polyQ protein. The present inventors observed a marked shift of soluble 60Q protein toward the lysosomal fraction of the cell lysates (FIGS. 2,E and F). These results suggested the efficient translocation of expanded polyQ by (R)HQ toward the lysosomes.

### EXAMPLE 4

polyQ Degradation Induced by RHQ Functions via a CMA-Dependent Mechanism

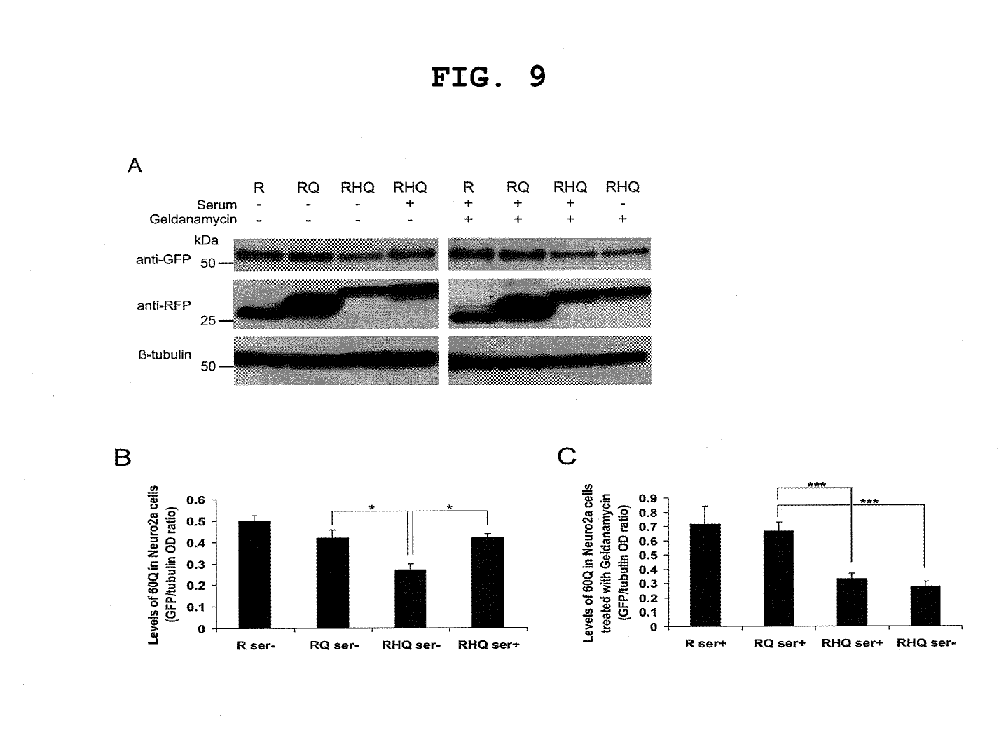

To further address the potential CMA-dependent mechanisms of the RHQ- and HQ-induced degradation, the present inventors silenced HSC70 and/or Lamp2a with shRNA (FIG. 8). In any knock-down combination, the effect of RHQ on polyQ aggregation in 150Q Neuro2a cells was inhibited (FIGS. 2,G and H), and RHQ and RQ had the same effect. To activate CMA, the present inventors incubated the cells with geldanamycin (P. F. Finn et al., Autophagy 1, 141 (2005)). Geldanamycin treatment of the 60Q Neuro2a cells enhanced the effect of RHQ on polyQ protein levels (FIG. 9). Geldanamycin mimicked the serum withdrawal condition in the presence of serum and RHQ (FIGS. 9,A and B), and serum withdrawal had no significant addition effect on the treatment with geldanamycin and RHQ (FIGS. 9,A and C). These observations suggest that a major portion of expanded polyQ was degraded by CMA.

### EXAMPLE 5

### Effect of RHQ in HD Model Mouse

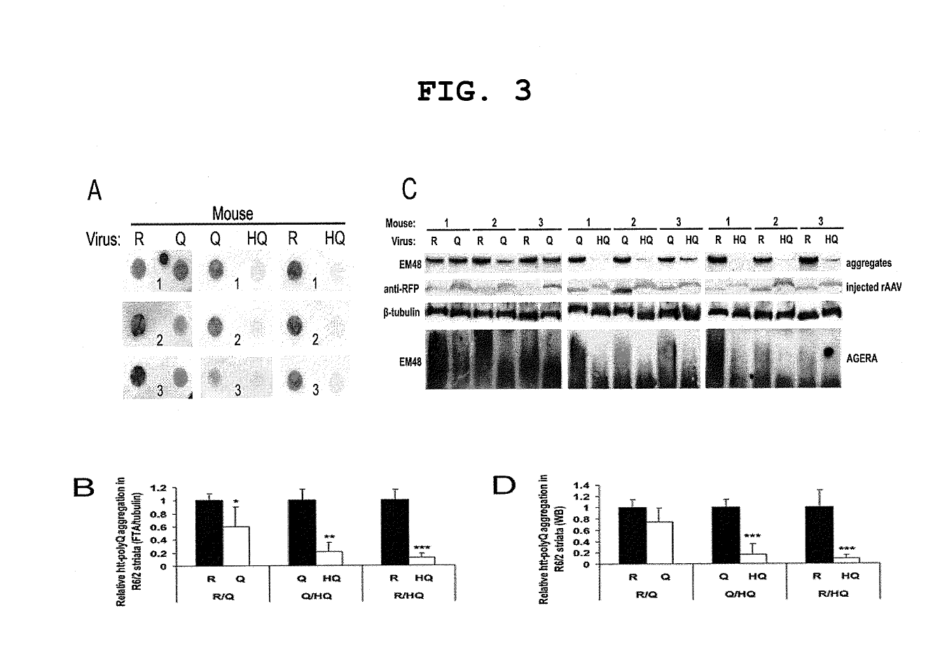

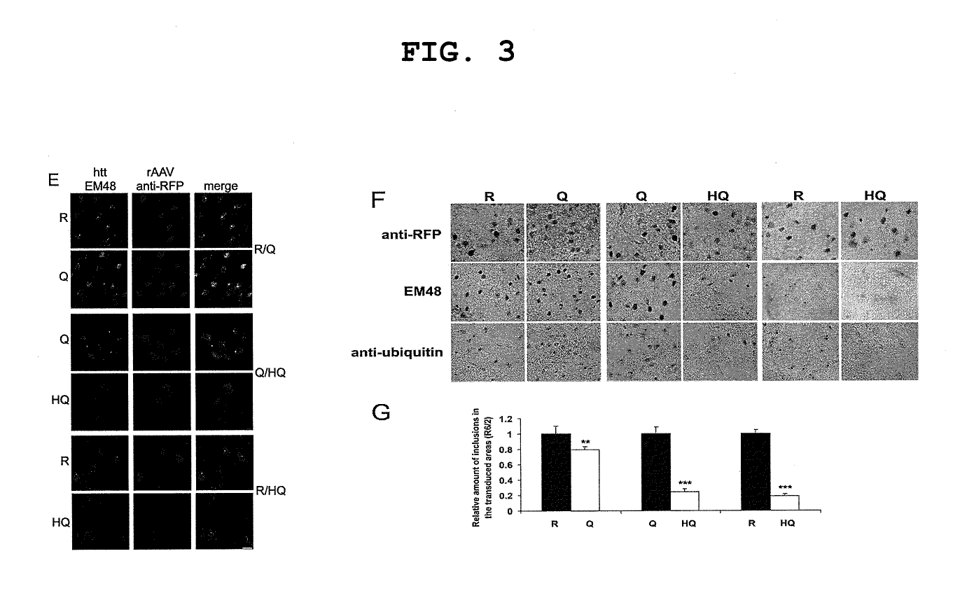

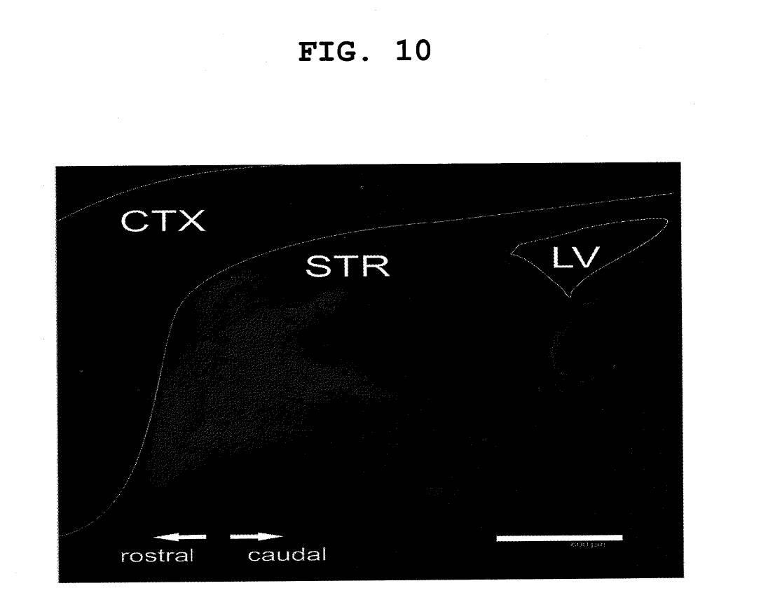

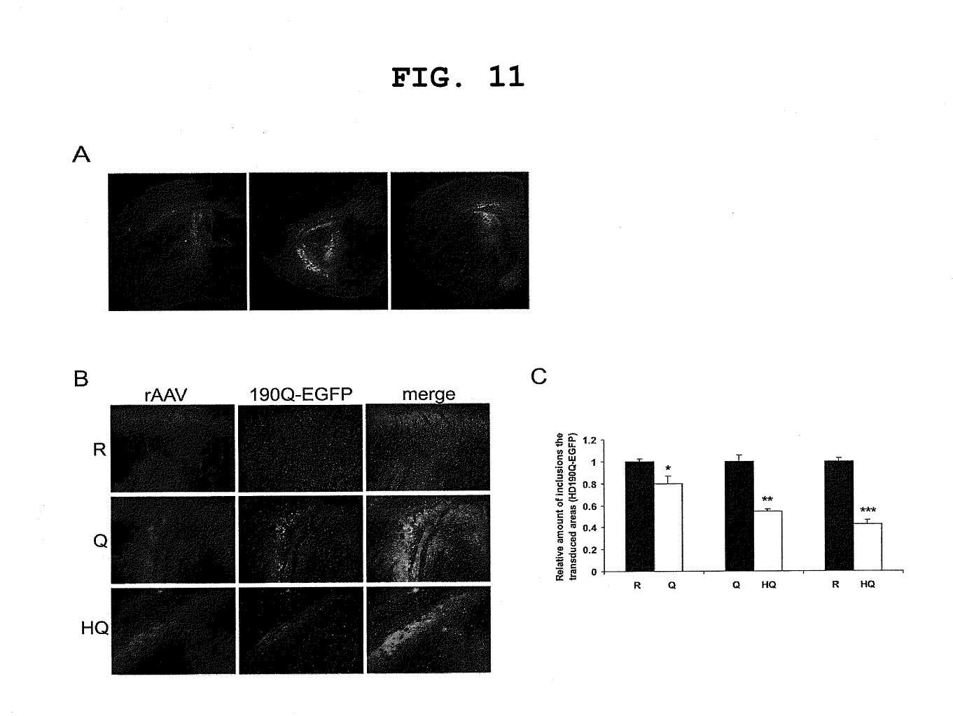

Next, the present inventors assessed the effect of the RHQ in two HD mouse models. Intrastriatal injections of recombinant adeno-associated virus (rAAV) encoding R, RQ or RHQ (rAAV-R, -Q, and-HQ) were performed in R6/2 mice at 4 weeks of age. The mice were injected in three different contralateral combinations (R/Q, Q/HQ or R/HQ), then the present inventors dissected the striata and prepared the lysates four weeks later. The rAAVs were widely distributed in the striatum (FIG. 10). The analysis of the SDS insoluble htt-polyQ aggregates by filter trap assay (FTA) revealed that the injection of rAAV-HQ reduced htt aggregation by 78.4% as compared to the contralaterally injected rAAV-Q, and by 87.2% as compared to rAAV-R in the R/HQ striata, while rAAV-Q reduced htt aggregation in the R/Q striata by 40% (FIGS. 3,A and B). The Western blot analysis of the lysates was consistent with the FTA results, where rAAV-Q decreased the aggregation by 25.6% in R/Q, rAAV-HQ by 83% in Q/HQ and by 90.8% in R/HQ brains (FIGS. 3,C and D). Furthermore, the AGERA method (described above in Experimental Procedures) revealed dramatic reduction of diffuse smearing staining, which suggested a higher molecular polyQ complex, in HQ but not in Q and R (FIG. 3C). Confocal microscopy of the brain sections showed extensive reduction of htt inclusions in the transduced cells with rAAV-HQ (FIG. 3E). When the present inventors counted the ubiquitin-positive inclusions in the transduced areas of the striata, a 21% decrease in inclusion number was observed in the rAAV-Q-injected site compared to the contralateral rAAV-R-injected site, while the rAAV-HQ injection reduced the amount of inclusions by 75.9% compared to rAAV-Q, and by 81.4% compared to rAAV-R (FIGS. 3,F and G). To test the effect of rAAV-HQ in another HD model, the present inventors injected the HD190Q-EGFP mice (S. Kotliarova et al., J Neurochem 93, 641 (2005)) at the age of 6 weeks and prepared the brain sections at 16 weeks (FIG. 11A). When the present inventors counted the EGFP-positive inclusions in the fresh frozen sections, the present inventors observed a reduction of 20.5% in R/Q, 45.8% in Q/HQ, and 57.3% in R/HQ brains (FIGS. 11,B and C). The results from two different HD mouse models clearly demonstrated the beneficial effect of HSC70bm because rAAV-HQ was able to decrease the expanded htt aggregation in vivo more efficiently than rAAV-Q.

### EXAMPLE 6

### Evaluation of the Therapeutic Potential of RHQ

The present inventors next evaluated the therapeutic potential of rAAV-HQ on the phenotype of R6/2 mice. Examination criteria consisted of body weight, clasping score, rotarod performance, and lifespan of mice injected at the age of four weeks in both striata with the same virus. The loss of body weight in both R/R and Q/Q mice was significantly more severe than that of the HQ/HQ mice, beginning at eight weeks (FIG. 4A). HQ/HQ mice exhibited significantly lower clasping scores at all time points from six to 12 weeks compared to both R/R and Q/Q mice, while in Q/Q mice, the limb clasping posture was ameliorated only at the ages of six and eight weeks (FIG. 4B). Consistent with the improvement in clasping scores, the present inventors also found that HQ/HQ mice showed significantly better performance than R/R or Q/Q mice on the rotarod at 6-12 weeks. In Q/Q mice, on the other hand, the latency to fall increased significantly only until the age of eight weeks (FIG. 4C). Most importantly, the lifespan of HQ/HQ mice increased markedly with a median survival time of 140 days compared to 108 days for R/R and 123 days for Q/Q mice (FIG. 4D), making the intrastriatal delivery of rAAV encoding HQ one of the most effective experimental therapy, to date, for increasing the lifespan of R6/2 mice (M. F. Beal, R. J. Ferrante, Nat Rev Neurosci 5, 373 (2004)).

To our knowledge, no single drug therapy has extended R6/2 lifespan more than our treatment, nor did RNAi treatment succeed in prolonging the lifespan of R6/2 mice to the extent reported here. Our results show that simply blocking htt aggregation, as suggested by (QBP1)₂ treatment, may not be sufficient to reduce polyQ toxicity. The soluble protein is probably more prone to degradation than the aggregated form, but such degradation may still be insufficient to keep the soluble protein from causing deterioration of affected cells. Our results suggest that by utilizing a linker molecule like (R)HQ, it is possible to specifically sort the abnormal protein to lysosomal degradation and decrease the overall levels of expanded polyQ in the cells through bulk degradation of the linked complex. This is an important discovery for future therapeutics for conformational diseases including not only polyglutamine diseases but also tauopathies and synucleopathies such as Parkinson's disease. The present inventors believe that with the discovery of peptides or intrabodies (intracytoplasmic antibodies) with even higher binding affinity to abnormal proteins, this strategy may be further applicable to other conformational diseases. For example, intrabodies which are able to inhibit aggregation and toxicity of α-synuclein have been extensively studied (S. Emadi et al., J Mol Biol 368, 1132 (2007), S. M. Lynch et al., J Mol Biol 377, 136 (2008)). To tag such an intrabody with HSC70bm might increase its therapeutic effect. Virus vectors may offer potential for future clinical use, although certain security concerns are a drawback of this therapeutic approach. The discovery or development of small compounds that are able to conjugate disease-related misfolded proteins with HSC70 may represent promising therapeutic solutions. The present inventors also believe that utilizing linker molecules similar to that presented herein would provide a novel method of therapeutic or experimental regulation of endogenous protein levels by enhancing their degradation.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

All of the references cited herein, including patents, patent applications, and publications, are hereby incorporated in their entireties by reference.

What is claimed is:

1. A method of degrading a target protein in a subject comprising administrating to the subject an effective amount of any of the following (a) and (b):
(a) a peptide comprising an HSC70-binding moiety and a target protein-binding moiety;
(b) a polynucleotide encoding the peptide of (a).

2. The method according to claim 1, wherein the HSC70-binding moiety comprises at least one motif capable of binding with HSC70.

3. The method according to claim 2, wherein the motif(s) is the amino acid sequence of SEQ ID NO:1 and/or the amino acid sequence of SEQ ID NO:2.

4. The method according to claim 1, wherein the target protein is an abnormal protein.

5. The method according to claim 4, wherein the abnormal protein is involved in a conformational disease.

6. The method according to claim 5, wherein the conformational disease is a polyglutamine disease.

7. The method according to claim 6, wherein the polyglutamine disease is Huntington's disease.

8. The method according to claim 1, wherein the target protein-binding moiety comprises a Polyglutamine-binding peptide 1 (QBP1).

9. The method according to claim 1, wherein the subject is a mammal.

10. An isolated peptide comprising an HSC70-binding moiety and a target protein-binding moiety, wherein the target protein is an abnormal protein.

11. An isolated polynucleotide encoding the peptide of claim 10.

12. The polynucleotide according to claim 11, wherein the abnormal protein is involved in a conformational disease.

13. The polynucleotide according to claim 12, wherein the conformational disease is a polyglutamine disease.

14. The polynucleotide according to claim 13, wherein the polyglutamine disease is Huntington's disease.

15. The polynucleotide according to claim 11, wherein the target protein-binding moiety comprises a Polyglutamine-binding peptide 1 (QBP1).

16. An expression vector comprising the polynucleotide of claim 11 operably linked to a promoter.

17. The vector according to claim 16, wherein the vector is a viral vector.


SEQUENCE LISTING (21 sequences)

SEQ ID NO: 1 - protein, 5 aa, Homo sapiens
KFERQ

SEQ ID NO: 2 - protein, 5 aa, Homo sapiens
VKKDQ

SEQ ID NO: 3 - protein, 5 aa, Homo sapiens
EFLKQ

SEQ ID NO: 4 - protein, 5 aa, Homo sapiens
QKVFD

SEQ ID NO: 5 - protein, 5 aa, Homo sapiens
QELRR

SEQ ID NO: 6 - protein, 5 aa, Homo sapiens
QEFIK

SEQ ID NO: 7 - protein, 11 aa, Artificial Sequence
  QBP1
SNWKWWPGIFD

SEQ ID NO: 8 - DNA, 132 bp, Artificial Sequence
  polynucleotide encoding peptide comprising HSC70bm and QBP1
atggcccgagttaagaaggatcaagctgagccgctgcaccgaaagttcgaacgtcaaccgcccgggtcgaactggaagtggtggccaggtatcttcgactcgaactggaagtggtggccaggtatcttcgac

SEQ ID NO: 9 - DNA, 15 bp, Artificial Sequence
  HSC70bm (1)
gttaagaaggatcaa

SEQ ID NO: 10 - DNA, 15 bp, Artificial Sequence
  HSC70bm (2)
aagttcgaacgtcaa

SEQ ID NO: 11 - DNA, 48 bp, Artificial Sequence
  Synthetic
gttaagaaggatcaagctggagccgctgcaccgaagttcgaacgtcaa

SEQ ID NO: 12 - protein, 23 aa, Artificial Sequence
  Synthetic
MWGWPNDFDWKGWFSPWKISWIN

SEQ ID NO: 13 - protein, 23 aa, Artificial Sequence
  Synthetic
MSNWKWWPGIFDSNWKWWPGIFD

SEQ ID NO: 14 - protein, 44 aa, Artificial Sequence
  Synthetic
MARVKKDQAEPLHRKFERQPPGWGWPNDFDWKGWFSPWKISWIN

SEQ ID NO: 15 - protein, 44 aa, Artificial Sequence
  Synthetic
MARVKKDQAEPLHRKFERQPPGSNWKWWPGIFDSNWKWWPGIFD

SEQ ID NO: 16 - DNA, 49 bp, Artificial Sequence
  forward primer
ccgaattcatgctagcaccatggatcctagtgtgattacatcagaaatg

SEQ ID NO: 17 - DNA, 45 bp, Artificial Sequence
  reverse primer
ttttagatcttatccggacttggcttcaaattcatacttggcgcc

SEQ ID NO: 18 - DNA, 33 bp, Artificial Sequence
  oligoDNA
gatctatacccatacgatgttccagattacgcg

SEQ ID NO: 19 - DNA, 33 bp, Artificial Sequence
  oligoDNA
gatccgcgtaatctggaacatcgtatgggtata

SEQ ID NO: 20 - DNA, 21 bp, Mus musculus
aaccattgcagtacctgacaa

SEQ ID NO: 21 - DNA, 21 bp, Mus musculus
aactggagaaagtctgcaacc